(19) United States
(12) Patent Application Publication
(10) Pub. No.: US 2016/0331407 A1
(43) Pub. Date: Nov. 17, 2016

(54) Evacuation Assist Device and Methods

(71) Applicant: LGO Medical, LLC, Franklin, WI (US)

(72) Inventor: Rebecca Anderson, Whitewater, WI (US)

(73) Assignee: LGO Medical, LLC, Franklin, WI (US)

(21) Appl. No.: 14/712,194

(22) Filed: May 14, 2015

(51) Int. Cl. A61B 17/42

(52) U.S. Cl. CPC A61B 17/42; A61B 2017/00424

(57) ABSTRACT

A device for human female vaginal wall self-manipulation is provided that assists in evacuation of the bladder and/or bowel. Methods are also provided in which the inventive evacuation device is utilized to evacuate the bladder and/or the bowel.

### FIELD OF THE INVENTION

The present invention relates to devices and methods for assisting with evacuating the bladder or bowel. More particularly, it relates to assisting such evacuation(s) in cases of female pelvic organ prolapse.

### BACKGROUND

A common problem for many women, especially but not exclusively those who have given birth vaginally, is pelvic-organ prolapse, or POP. For example, a uterine prolapse caused directly by the trauma and stretching of labor and vaginal childbirth can lead to secondary conditions including, but not limited to, a cystocele (prolapsed bladder pressing against the front vaginal wall) and a rectocele (prolapsed rectum pressing against the rear vaginal wall). These conditions can lead to urinary incontinence, fecal incontinence, difficulty urinating, and/or constipation.

In such cases, inserting one's fingers vaginally to apply pressure to the vaginal wall at the location of the cystocele or rectocele can assist with evacuation. However, this can be an awkward, unsanitary and unpleasant procedure.

Vaginal devices such as pessaries have long been used to relieve or treat long-term effects of pelvic organ prolapse. However, when in use, such devices are entirely inside the vaginal canal, and consequently cannot be easily manipulated to apply sufficient pressure to assist in defecation or urination. Furthermore, such a device having sufficient girth to apply such pressure continuously would be uncomfortable or even harmful if left inserted for long periods.

Insertable devices with an inflatable or expandable inserted end are also known for assisting with evacuation. However, a drawback of such insertable devices compared to the finger insertion method is that a user has only limited control, if any, of the location and direction of pressure applied by the inflatable or expandable end. In addition, the structural complexity of such devices makes them difficult to clean and thus potentially unsanitary, so that an inconvenient and wasteful disposable sleeve is required to be placed over their inserted ends before each use and then discarded after use.

A need therefore exists for a comfortable, ergonomic, sanitary, and portable device and method for assisting with bladder or bowel evacuation in a woman with a cystocele and/or a rectocele.

### SUMMARY OF THE INVENTION

In accordance with one aspect of the present invention, a device for a human female vaginal wall self-manipulation is provided. The device is particularly useful for assisting with evacuation of the bladder or bowel and particularly for assisting such evacuation in cases of female pelvic organ prolapse, for example. In accordance with one embodiment, the device includes a vaginally insertable portion having a terminal bulbous tip at the end of an insertable shaft having two ends and an elongate, bent shape is connected at one end thereof to the insertable portion and a handle is connected to the other end of the stem so that a human female may grasp and manipulate the handle to press the tip against a portion of the vaginal wall to displace a portion of an organ pressing against the portion of the vaginal wall. The insertable portion, stem and handle collectively form a stiff elongate tool body.

In accordance with another embodiment of the device in accordance with the invention, the insertable shaft is generally straight.

Typically, the device will be constructed of a medical grade material. Such materials are well known to those skilled in the art and may be selected from the group that includes polyethylene, polypropylene, silicone rubber and stainless steel, for example.

In accordance with another aspect of the invention, a portion of the outer surface of the terminal bulbous tip is convex with substantially circular cross sections generally centered on a longitudinal axis of the terminal bulbous tip. The terminal bulbous tip may have an axis that is substantially aligned with an axis of the insertable shaft. In addition, the bulbous tip axis may be oblique to an axis of the insertable shaft.

In accordance with another aspect of the invention, the terminal bulbous tip may include an outer surface portion for contacting the portion of the vaginal wall that is perpendicularly displaced from an axis of the insertable shaft by a contact radius, the contact radius being at least about 50% larger than a radius or an effective radius of the shaft. As used herein, effective radius means the largest cross-sectional radial dimension from or normal to the longitudinal axis, respectively, of each of the insertable shaft and bulbous tip elements, respectively.

In one embodiment, the tool body can be integrally formed and thus constitutes an integral device, being a unitary member. Such construction facilitates cleaning of the device to help minimize infection risks. In accordance with another embodiment of the invention, portions of the device may be detachably connected to facilitate compact storage of the device when in a disassembled state. For example, the handle can be detachably connected to the stem and the tip can be detachably connected to the stem. In addition, the device may comprise a kit in which a plurality of interchangeable terminal bulbous tips are provided of different shape and/or size.

In accordance with another aspect of the invention, the terminal bulbous tip may be integrally formed as part of an insertion member that includes the bulbous tip at its insertable end and at least a portion of the insertable shaft of the device is integrally formed with the bulbous tip. The insertion member may then be detachably connected to the stem at a connecting end opposite the insertable end of the insertable shaft.

Typically, the length of the insertable portion to the leading end of the bulbous tip is in the range of from about 6 inches to about 10 inches.

In accordance with another embodiment of the invention, the stem is bent at an angle of from about 45° to about 85° and typically the distance between the handle and the terminal portion of the terminal bulbous tip is typically from about 10 inches to about 16 inches. It is to be understood that other angles for the bent stem and the distance between the handle and terminal portion of the bulbous tip may be utilized, if desired. In addition, the nature of the bent stem can also generally be as desired, for example, ranging from a gradual curve (as shown in FIG. 1) to a straight line geometric angle or angles, i.e., composed of the intersection of two or more straight or approximately straight lines or members. Thus, the bent stem also could be composed of a series of relatively straight segments or members joined at desired angles to produce the desired overall bent shape.

In accordance with another aspect of the invention, a self-assisted method of evacuating a bladder or bowel of a human female is provided. The method includes using a vaginal wall manipulation device as previously described in which the human female vaginally self-inserts the insertable portion of the device and thereafter the human female grasps a portion of the device and manipulates the device to press the bulbous tip against the vaginal wall to assist in evacuating the bladder or the bowel of the human female. Typically, the grasping a portion of the device located exteriorly of the vagina and manipulating the device to press the tip against the vaginal wall comprises compressing the tip against a cystocele or a rectocele in the vaginal wall. The urination or defecation can occur while keeping the tip pressed against the relevant portion of the vaginal wall, i.e., either against the cystocele or a rectocele, for example.

The method may further include connecting the bulbous tip to the stem before the vaginal self-inserting of the insertable portion of the device occurs. The method may still further include connecting a handle to the stem before the manipulating of the device. The self-inserting of the insertable portion of the device may also include inserting at least part of the stem of the device into the vagina.

### BRIEF DESCRIPTION OF THE DRAWINGS

FIG. 1 is a perspective view of a device in accordance with the invention;

FIG. 2A is a perspective view of an alternate embodiment of the present invention;

FIG. 2B is a plan view of an alternate embodiment of the insertion member;

FIG. 2C is a plan view of another embodiment of the insertion member;

FIG. 3A is an exploded perspective view of the embodiment of FIG. 2;

FIG. 3B illustrates an end elevation view of a portion of the device of FIG. 3A;

FIG. 3C is a fragmentary sectional view showing an alternate embodiment for attachment of an insertion member to a stem member;

FIG. 4 is a top elevation view of one embodiment of a portion of the inventive device;

FIG. 5 is a top elevation view of another embodiment of a portion of the inventive device;

FIG. 6 is a schematic view of relevant portions of a human female anatomy;

FIG. 7 illustrates use of the inventive device on the female anatomy shown in FIG. 6; and

FIG. 8 illustrates use of the inventive device for evacuating a bowel.

### DETAILED DESCRIPTION OF THE INVENTION

Vaginally insertable devices and methods of using them in self-assisted evacuation according to the present invention will now be described in detail.

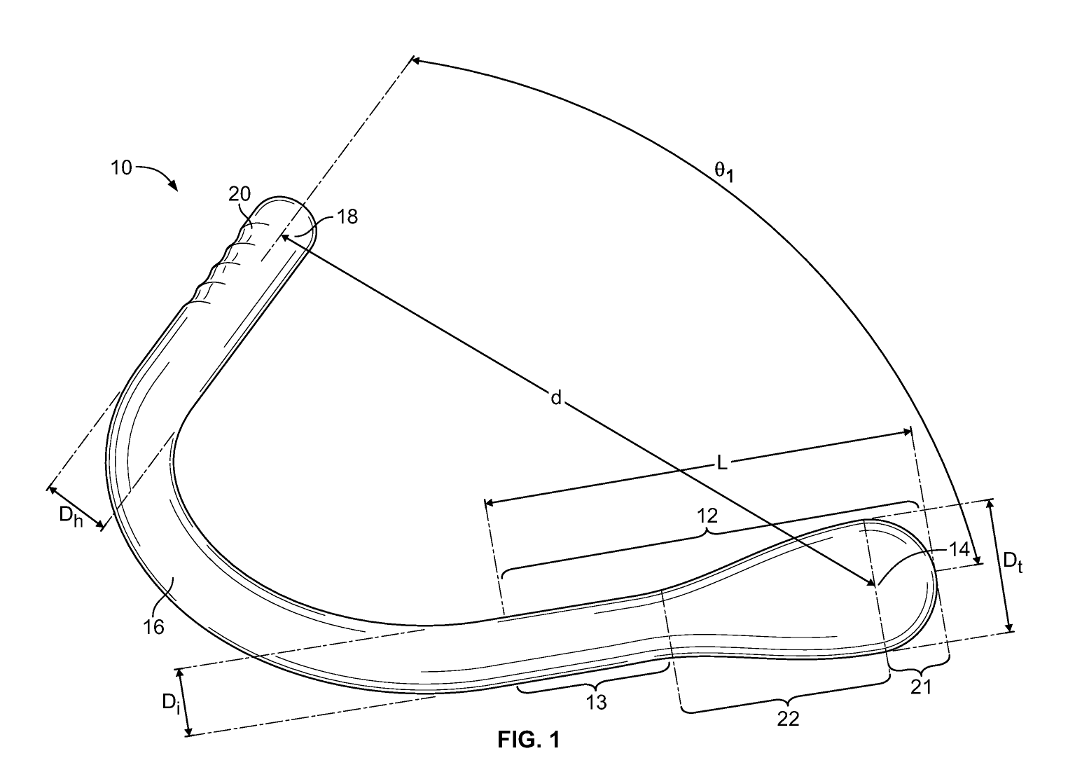

With reference to FIG. 1, a first embodiment of the invention is disclosed, which is a one-piece self-assisted evacuation device 10 shown in perspective. Device 10 is a unitary, generally V- or U-shaped body that includes a vaginally insertable portion 12 with a generally straight shaft 13 and a tear-drop shaped bulbous tip 14 at its insertable end, a curved stem portion 16 having one end adjacent insertable portion 12 and another end adjacent a handle portion 18 comprising finger-grip depressions 20. Other shapes for bulbous tip 14 may be utilized as desired. Shaft 13 may have a cross-section that is circular, oval, elliptical or some other shape as desired and typically having a smoothly curved surface without a rough or sharply edged shape or surface. Advantageously, the one-piece, seamless form of device 10 avoids presenting any rough surfaces or sharp edges on insertable portion 12, as well as facilitating easy lubrication and cleaning of its outer surface. The longitudinal axis of device 10 (and of device 23 with tip 26) including the longitudinal axis of insertable portion 12, stem portion 16 and handle portion 18, lie in a single plane.

Device 10 is also characterized by certain shapes and dimensions of its component features, including those labeled in FIG. 1. Thus, handle portion 18 is displaced from tip 14 by a distance d and offset by an angle θ₁. Distance d, the distance from the approximate center of the handle at its longitudinal midpoint to the approximate center of bulbous tip 14 at its approximate largest diameter or midpoint, should be long enough to provide a clearance between handle portion 18 and the front of a woman's body when insertable portion 12 is inserted, and distance d and angle θ₁ should provide a comfortable and ergonomic hand position on handle portion 18. A preferred or typical value of distance d may be within a range, and thus different sizes of device 10 may be provided to accommodate women of different heights, body sizes, and body shapes. In one example, distance d is about 11 inches, and angle θ₁ is about 53°. A length L typically in the range of from about 6 inches to about 10 inches of insertable portion 12, that is, a distance from the insertable end of tip 14 to the end of shaft 13, where it transitions into curved stem portion 16, is preferably sufficient to permit straight insertion of tip 14 to a depth of the vaginal canal corresponding to the position of the cystocele or rectocele, but no longer than the depth from the vulva to the cervix. A typical length of insertable portion 12 may be 6 inches.

A maximum tip diameter Dₜ of tip 14 is advantageously as large as comfort and physiology permits, to reduce the tendency of a cystocele or rectocele to slide past tip 14 to its right or left side. On the other hand, a diameter Dᵢ of shaft 13 (or effective diameter of a non-circular shaft) is preferably much smaller than that of tip 14, to maximize the amount of lateral pressure or displacement that may be applied by tip 14 without shaft 13 running into the vaginal wall or other obstruction, possibly applying pressure where it may not be desired, or perhaps even counterproductively interfering with evacuation. Thus, in one embodiment, Dᵢ is about 21 mm and Dₜ is up to about 44 mm, or about 110% larger than Dᵢ. On the other hand, to provide a comfortable grip, it may be desirable for a diameter Dₕ of handle portion 18, to be slightly larger than insertable shaft diameter Dᵢ, such as where Dᵢ is about 21 mm+/−10 mm and Dₕ is about 24+/−10 mm, and stem portion 16 may be tapered so that its diameter at an approximate midpoint along the length of stem portion 16 is about 22 mm+/−10 mm.

In a broader sense, an “effective radius” of the tip, which may be defined for purposes of this description as a greatest radial distance from the axis of the shaft to a portion of the outer surface of the tip, in effect an approximate measure of the lateral depth to which the tip may displace the vaginal wall, and thus the cystocele or rectocele, before the shaft begins to interfere, should be significantly larger than the radius of the shaft. For example, the effective radius of the tip typically may be between about 20% and about 110% larger, or in absolute terms about 2-11 mm larger, than the radius of the shaft. The shape of tip 14 is depicted as that of a tear drop, including a generally hemispherical insertable end portion 21 and a gradually tapered base portion 22, but many variations are possible within the scope of the invention, including but not limited to shapes that more closely resemble a sphere, an elliptical or oval cross-sectional shape as well as shapes having an approximately uniform maximum cross-sectional breadth or girth along a straight portion of their length, rather than transitioning directly from an arc to a taper at a localized maximum cross section, for example. In some cases, a front and rear side of an insertable tip may advantageously have a reduced curvature or even present a flat or a relatively flat face, again limiting the tendency of the cystocele to slip around one side of the tip. In addition, flattening of the front and rear faces of the tip may advantageously permit its frontal/coronal profile to be broadened without a net increase in perimeter. Stem portion 16 may also advantageously have a different shape than that depicted. For example, straightening the curved region of stem portion 16, to the extent possible without unduly limiting the insertable depth of insertable portion 12 or otherwise impinging on the woman's body during use, would make device 10 slightly more compact for storage or carrying.

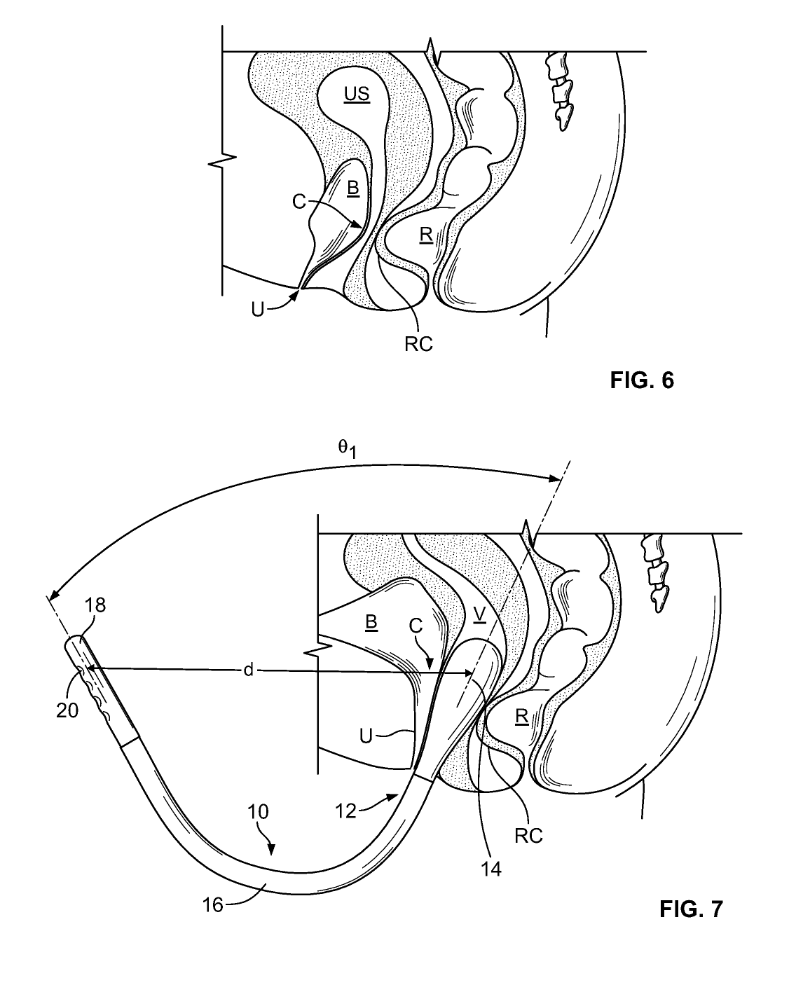

Aspects of using devices in accordance with the invention are illustrated in FIGS. 6-8, where like reference numerals and letters indicate like elements or structures. As shown in FIGS. 6-8, the female anatomy includes a vagina V, a bladder B, a rectum R, a urethra U, a uterus US, a cystocele C and a rectocele RC. In operation of device 10, a user/patient applies a suitable personal lubricant as desired to insertable portion 12, positions herself to evacuate, for example over a toilet, vaginally self inserts insertable portion 12, grasps handle portion 18, and manipulates handle portion 18 to move tip 14 against a rectocele (if defecating), cystocele (if urinating), or both, either before commencing or during evacuation. In some cases, the size and shape of tip 14 may provide sufficient pressure or displacement of the cystocele, rectocele, or both, to promote comfort or ease of urination or defecation, simply upon straight insertion of insertable portion 12 with its axis generally aligned with the vaginal canal. Otherwise, the user/patient may use handle portion 18 to manipulate tip 14 against the cystocele C or rectocele RC until evacuation becomes easier or more comfortable.

For example, the user/patient may push handle portion 18 forward in a direction away from her belly and/or tilt handle portion 18 forward in her body's sagittal plane to apply or increase displacement or pressure of tip 14 against a portion of the anterior vaginal wall that abuts cystocele C, and/or to remove or decrease displacement or pressure of tip 14 applied against a portion of the posterior vaginal wall that abuts a rectocele. Conversely, rearward pulling or tilting of handle 18 may apply or increase pressure or displacement of tip 14 against the posterior vaginal wall, where it abuts a rectocele, or it may remove or decrease similar pressure or displacement of tip 14 applied against the anterior vaginal wall where it abuts a cystocele. Other modes of manipulation of device 10 are also possible. For example, to control the angle in her body's transverse/horizontal plane at which pressure or displacement is applied when handle 18 is pushed, pulled, or tilted, a user/patient may first move handle 18 to the right or left generally along an arc centered on the axis of insertable portion 12 before pushing, pulling, or tilting. Rotating her wrist to the left or right will generally lean handle portion 18 to the left or right in her body's frontal/coronal plane, and insertable portion 12 will generally mirror the left or right leaning of handle portion 18, generally resulting in left or right tilting of tip 14 accompanied by lateral displacement of tip 14 in the same direction.

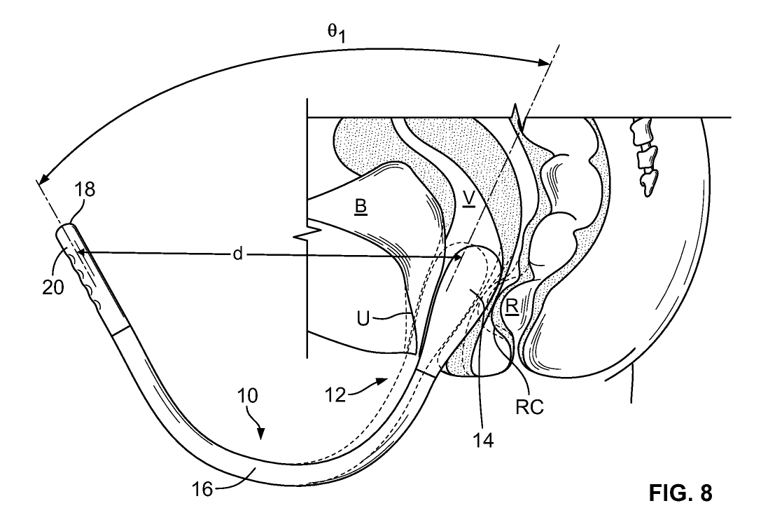

While evacuating, the user may reposition tip 14 against the rectocele or cystocele as needed to maintain ease and comfort of evacuation. For example, she may feel the cystocele or rectocele slide to the right or left around tip 14 so that it ceases to be braced against tip 14, accompanied by a return of discomfort or strain. In this case, the user/patient may, for example, move handle portion 18 to the right or left to reorient the transverse/horizontal angle of applied pressure to a direction that points generally toward the middle of the cystocele or rectocele, or she may rotate her wrist to the right or left to tilt and displace tip 14 along the vaginal wall to the position where the cystocele or rectocele abuts it, until relief is provided. When evacuation is complete, the user/patient removes insertable portion 12 and cleans its outer surface, optionally using a cleaning wipe (not shown) which may be provided in a pouch or kit (not shown) with device 10. Examples of methods in accordance with the invention are illustrated in FIGS. 6-8, wherein device 10 is shown to be employed to push a cystocele C of a bladder B, shown in FIGS. 6-7, away from the front wall of a vaginal canal V and to push a rectocele R of a bowel B, shown in FIG. 8, away from the vaginal canal V. Device 23, described below, can be similarly used.

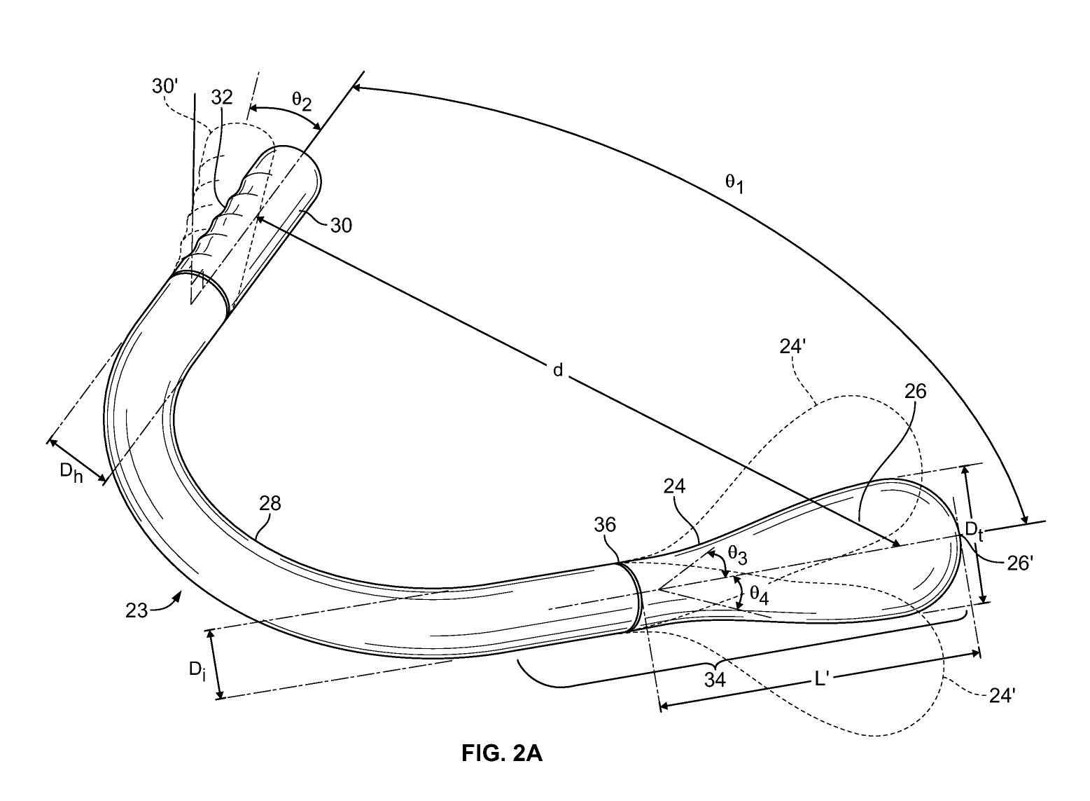

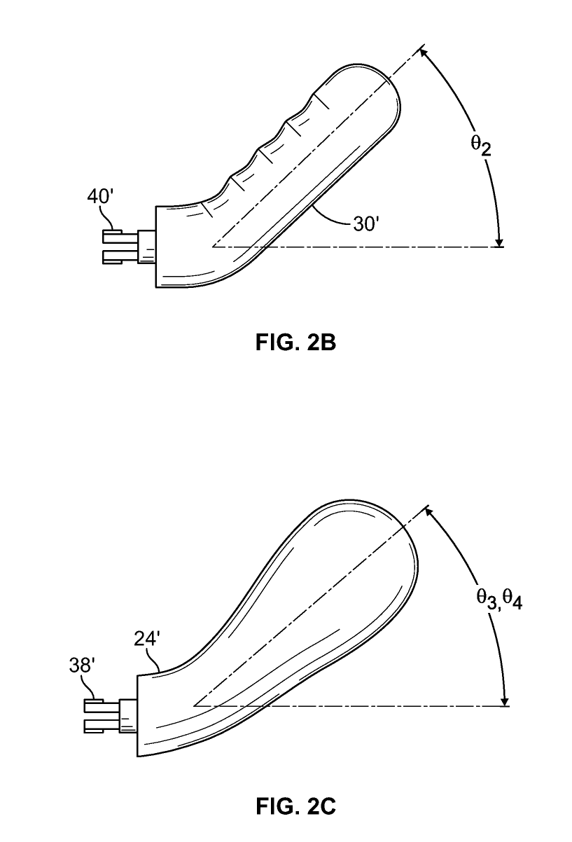

With reference to FIGS. 2A-5, a modular self-assisted evacuation device 23 according to another embodiment of the invention is illustrated. Device 23 includes a vaginal insertion member 24 with a tear-drop shaped bulbous tip 26 at its insertable end, a stem member 28 having one end removably connected to insertion member 24 and another end removably connected to a handle member 30 comprising finger-grip depressions 32. Insertion member 24 may correspond to part or all of an insertable portion 34 (typically about 6-10 inches in length) of device 23, depending on the location of an insertion member joint 36 by which insertion member 24 is connected to stem member 28. Thus, in the illustrated example, insertable portion 34 includes all of insertion member 24 and part of stem member 28. The length L′ from the inventive member joint 36 to the end 26′ of bulbous tip 26 is typically in the range of from about 2.5 to about 5.0 inches.

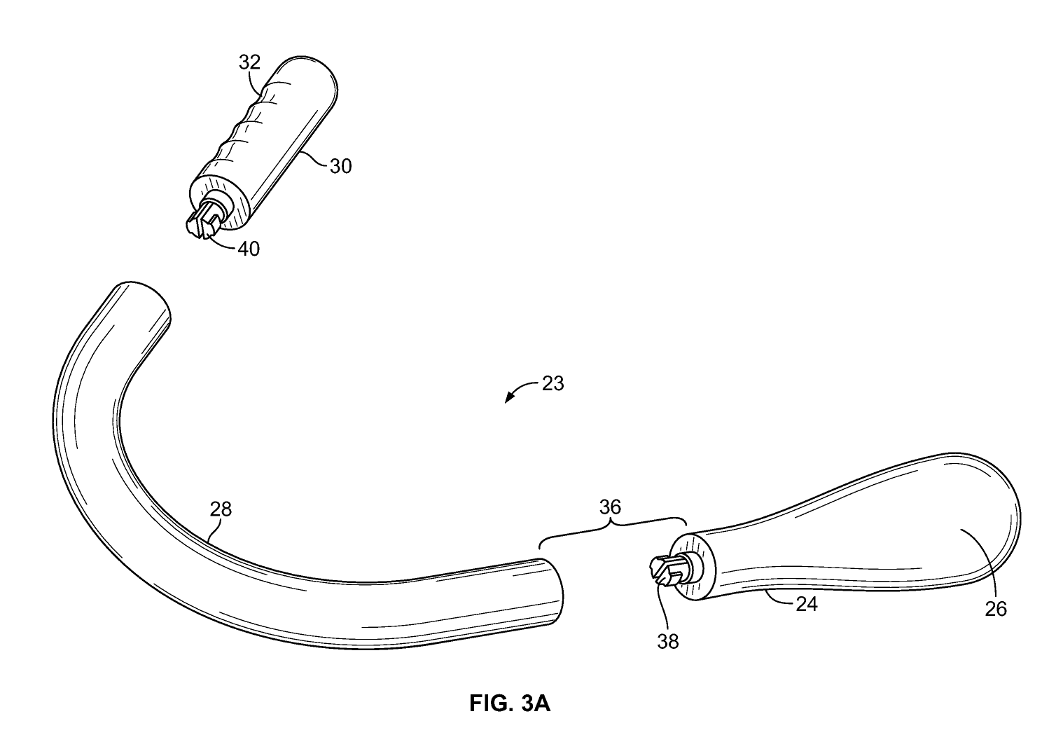

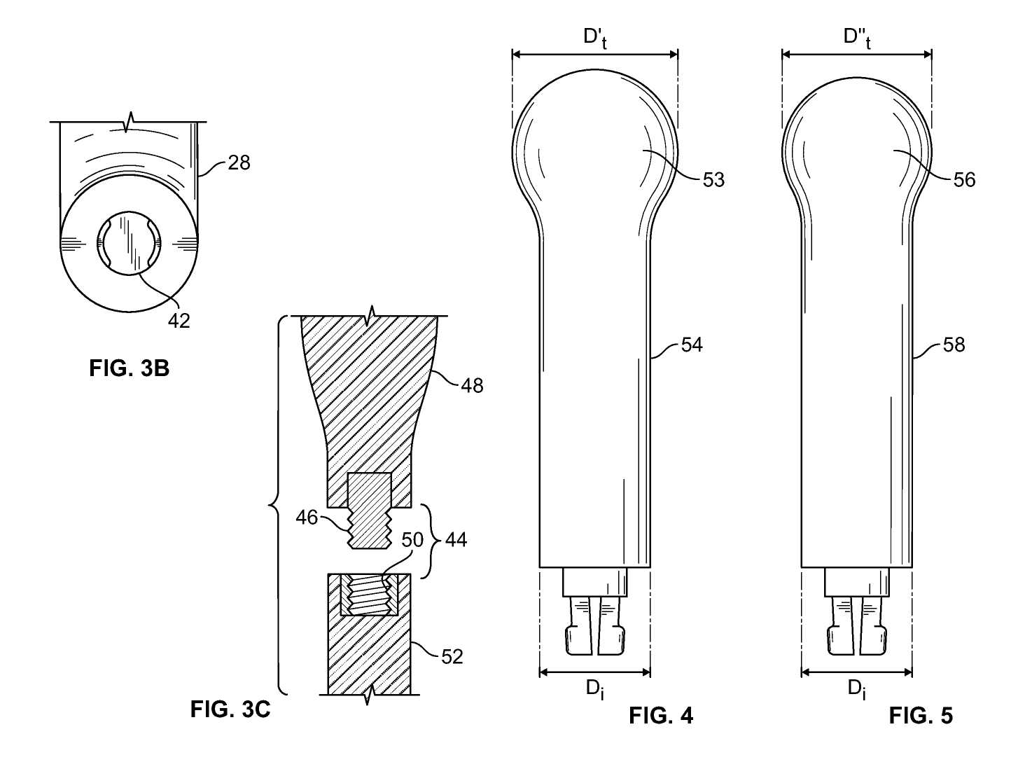

Examples of joints suitable for connecting the components of modular device 23 are illustrated in FIGS. 3A-3C. FIG. 3A is an exploded perspective view of device 23 illustrating generally T-shaped male lugs 38, 40 associated with insertion member 24 and handle member 30, respectively. Lugs 38, 40 are adapted to insert and twist about 90° to lock into a female connector 42 as illustrated in FIG. 3B, which may represent either the insertable end or the handle end of stem member 28. FIG. 3C illustrates an alternative joint 44, by which a male threaded connector 46 of an insertion member 48 is threaded into a female threaded connector 50 of a stem member 52, which may also be provided as a handle joint (not shown). A threaded joint such as joint 44 may have the advantage of being sturdier than a twist-lock joint, particularly if threaded connectors 46, 50 are formed of a stronger material (such as stainless steel) than that of the main components of a self-assisted evacuation device according to the invention, which are typically made of a medical grade polymer such as a high density polyethylene or polypropylene, for example. On the other hand, a twist-lock joint as illustrated in FIG. 3A may advantageously permit a user/patient to attach a handle member or an insertion member in a selected one of a plurality of orientations. For example, to lock handle member 30 in place, a right-handed user/patient may prefer to twist finger-grip depressions 32 to the left-facing position illustrated in FIG. 2A, whereas a left-handed user/patient may prefer to twist depressions 32 to a right-facing position. Further, with reference to FIGS. 2A and 2B, if a handle member 30′ has a bent shape (so as to be inclined out of the plane shared by stem member 28 and insertion member 24 by an angle θ₂ as shown in FIG. 2A), instead of the generally axisymmetric shape depicted for handle member 30, a right-handed user/patient may choose to twist handle member 30′ to lean left when locking it in place with a corresponding male lug 40′, whereas a left-handed user/patient may choose to twist handle member 30′ to lean right. Likewise, with reference to FIGS. 2A and 2C, if an insertion member 24′ has a bent shape, a user/patient with a cystocele may choose to twist insertion member 24′ so that tip 26′ leans forwardly by a sagittal angle θ₃, a user/patient with a rectocele may choose to twist insertion member 24′ so that a tip 26′ leans rearwardly by a sagittal angle θ₄, and a user/patient having both a cystocele and a rectocele may twist insertion member 24′ to lean in the appropriate direction to better assist with defecation or urination as needed.

Advantageously, modular device 23 may be taken apart and thus made more compact for storage or carrying. In addition, and especially with regard to tip 26, flexibility is provided to substitute a selected one of a plurality of interchangeable components. Thus, women with different anatomies, or the same woman on different days or at different stages of progression of, or recovery from, a cystocele or rectocele, may select an insertion member having a different tip size or shape. For example, in addition to the tear-drop shaped tip 26, a large spherical tip 53 of an insertion member 54, having a maximum tip diameter D′ₜ, or a small spherical tip 56 of an insertion member 58, having a maximum tip diameter D″ₜ, may be selected, as shown in FIGS. 4 and 5 respectively.

While the invention has been described with respect to certain embodiments, as will be appreciated by those skilled in the art, it is to be understood that the invention is capable of numerous changes, modifications and rearrangements, and such changes, modifications and rearrangements are intended to be covered by the following claims.

What is claimed is:

1. A device for vaginal wall self-manipulation comprising
a vaginally insertable portion comprising a terminal bulbous tip at an insertable end of an insertable shaft;
a stem having an elongate, bent shape connected at one end to the insertable portion; and
a handle connected to the stem so that a human female may grasp and manipulate the handle to press the tip against a portion of the vaginal wall to displace a portion of an organ pressing against the portion of the vaginal wall;
the insertable portion, stem, and handle collectively forming a stiff elongate tool body.

2. The device of claim 1, wherein the insertable shaft is generally straight.

3. The device of claim 1, wherein the vaginally insertable portion is composed of a medical grade material.

4. The device of claim 3, wherein the material is selected from the group consisting of polyethylene, polypropylene, silicone rubber, stainless steel.

5. The device of claim 1, wherein a portion of an outer surface of said tip is convex with substantially circular cross sections generally centered on a longitudinal axis of the tip.

6. The device of claim 5, said tip having an axis that is substantially aligned with an axis of the insertable shaft.

7. The device of claim 5, said tip axis being oblique to an axis of the insertable shaft.

8. The device of claim 1, wherein the tip includes an outer surface portion for contacting said portion of the vaginal wall that is perpendicularly displaced from an axis of the insertable shaft by a contact radius, the contact radius being at least about 50% larger than the effective radius of the shaft.

9. The device of claim 1, said tool body being integrally formed as a unitary member.

10. The device of claim 1, said handle being detachably connected to the stem.

11. The device of claim 1, said tip being detachably connected to the stem.

12. The device of claim 11, further comprising a plurality of interchangeable tips, each of said tips being configured to be connected to the stem.

13. The device of claim 11, said tip being integrally formed in an insertion member comprising the tip at its insertable end and at least a portion of the insertable shaft, the insertion member being detachably connected to the stem at a connecting end opposite the insertable end.

14. The device of claim 1, a length of the insertable portion from the connecting end is from about 6 inches to about 10 inches.

15. The device of claim 1, the stem being bent at an angle of from about 45° to about 85°.

16. The device of claim 1, wherein the distance between the handle and the tip is from about 10 inches to about 16 inches.

17. A self-assisted method of evacuating a bladder of a human female using a vaginal wall manipulation device comprising a vaginally insertable portion comprising a terminal bulbous tip at an insertable end of an insertable shaft, a stem having an elongate, bent shape connected at one end to the insertable portion; and a handle connected to the stem so that a human female may grasp the handle to press the tip against a portion of the vaginal wall to displace a portion of an organ pressing against the portion of the vaginal wall, the method comprising:
the human female vaginally self-inserting the insertable portion of the device;
the human female grasping a portion of the device and manipulating the device to press the tip against the vaginal wall to assist in evacuating the bladder of the human female.

18. The method of claim 17 wherein the grasping a portion of the device located exteriorly of the vagina and manipulating the device to press the tip against the vaginal wall to assist in evacuating the human female's bladder comprises pressing the tip against a cystocele in the vaginal wall.

19. The method of claim 17, said urinating comprising urinating while keeping the tip pressed against the vaginal wall.

20. The method of claim 17, further comprising connecting said tip to said stem before said vaginally self-inserting the insertable portion of the device.

21. The method of claim 19, said self-inserting the insertable portion of the device comprising inserting at least part of the stem of the device.

22. The method of claim 17, further comprising connecting said handle to said stem before said manipulating of the device.

23. A self-assisted method of evacuating a bowel of a human female using a vaginal wall manipulation device comprising a vaginally insertable portion comprising a terminal bulbous tip at an insertable end of an insertable shaft, a stem having an elongate, bent shape connected at one end to the insertable portion; and a handle connected to the stem so that a human female may grasp the handle to press the tip against a portion of the vaginal wall to displace a portion of an organ pressing against the portion of the vaginal wall, the method comprising:
the human female vaginally self-inserting the insertable portion of the device;
the human female grasping a portion of the device and manipulating the device to press the tip against the vaginal wall to assist in evacuating the rectum of the human female.

24. The method of claim 23 wherein the grasping a portion of the device located exteriorly of the vagina and manipulating the device to press the tip against the vaginal wall to assist in evacuating the human female's rectum comprises pressing the tip against a rectocele in the vaginal wall.

25. The method of claim 23, further comprising defecating while keeping the tip pressed against the vaginal wall.

26. The method of claim 23, further comprising connecting said tip to said stem before said vaginally self-inserting the insertable portion of the device.
